(19) United States
(12) Patent Application Publication
(10) Pub. No.: US 2009/0253769 A1
(43) Pub. Date: Oct. 8, 2009

(54) Inhibitors of mammalian hdac 11 useful for treating hdac 11 mediated disorders

(76) Inventors: Gessica Filocamo, Rome (IT); Christian Steinkuhler, Rome (IT)

Correspondence Address: MERCK AND CO., INC, P O BOX 2000, RAHWAY, NJ, 07065-0907, US

(21) Appl. No.: 10/586,701

(22) PCT Filed: Jan. 18, 2005
(86) PCT No.: PCT/EP2005/000559

§ 371 Date: Jul. 20, 2006

Related U.S. Application Data
(60) Provisional application No. 60/537,940, filed on Jan. 21, 2004.

(51) Int. Cl. A61K 31/713; C07H 21/02; C12N 5/00; C12Q 1/68; A61P 35/00

(52) U.S. Cl. 514/44 A; 536/24.5; 435/375; 435/377; 435/6

(57) ABSTRACT

The present invention, relies, in part, on the discovery of the role of HDAC 11 in various cell proliferative disorders including cancer. In the main, the invention provides for methods of inhibiting HDAC 11 as a means of treating cell proliferative disorders. Reagents for use in inhibiting human HDAC 11 are also provided.

### BACKGROUND OF THE INVENTION

This invention relates to histone deacetylases, in particular HDAC 11, its role in cellular proliferative diseases such as cancer and methods of treating said disorders via the use of newly identified HDAC 11 inhibitors.

In eukaryotic cells, the orderly packaging of DNA in the nucleus plays an important role in the regulation of gene transcription. In the resting cell, DNA is tightly compacted to prevent transcription factor accessibility.

The compact complex termed chromatic results from the tight association of nuclear DNA with histones. The basic repeating unit in chromatin is the nucleosome, which consists of 146 bases of DNA wrapped around a complex of eight histone proteins, two molecules each of the core histones, H2A, H2B, H3, and H4. Each core histone octamer is comprised of several highly conserved structural motifs including a globular domain and an N-terminal tail domain that extends outside of the nucleosome. Importantly, the packaging of DNA into nucleosomes acts as a barrier to the initiation of transcription by preventing the access of transcription factors, and RNA polymerase II, to their cognate recognition sequences (Workman, J. L. & A. R. Buchman (1993) Trends. Biochem. Sci. 18:90-95).

During activation of the cell this compact, inaccessible DNA is made available to DNA-binding proteins, thus allowing the induction of gene transcription (Beato, M. (1996) J. Mol. Med. 74:711-724; Wolffe, A. P. (1997) Nature 387:16-17). The physical interaction between the core histone particle and DNA principally occurs through the negatively charged phosphate groups of the DNA and the basic amino acid moieties of the histone proteins. These histone N-terminal tails are enriched in basic amino acids that are sites for post-transcriptional modifications, and are thought to mediate histone-DNA contacts through electrostatic interactions with DNA's negatively charged phosphate backbone. (Beato, M (1996) J. Mol. Med. 74:711-724; Beato, M. & K. Eisfeld (1997) Nucleic. Acids. Res. 25:3559-3563). Core histones may be modified by acetylation, phosphorylation, methylation, ADP ribosylation or ubiquitinylation of specific amino acid residues (Wu, R. S et al (1986) CRC Crit. Rev. Biochem. 20:201-263).

There is a vast body of evidence suggesting that increased gene transcription is associated with an increase in histone acetylation, whereas hypo-acetylation is correlated with reduced transcription or gene silencing (Ura, K et al (1997) EMBO J. 16:2096-2107; Wolffe, A. P (1997) Nature 387:16-17).

Histone acetylation, a reversible modification, occurs on actively transcribed chromatin via the action of histone acetyl transferases (HATs) (Perry, M. & R. Chalkley (1982) J. Biol. Chem. 257:7336-7347), while histone deacetylation is catalyzed by a family of enzymes termed histone deacetylases (HDACs), which serve to repress gene expression. See, for example, Grunstein, Nature 389, 349-352 (1997); Pazin et al., Cell 89, 325-328 (1997); Wade et al., Trends Biochem. Sci. 22, 128-132 (1997); and Wolffe, Science 272, 371-372 (1996). Essentially, acetylation of histones reduces their positive charge, thereby relaxing the structure of the nucleosome and facilitating the interaction of transcription factors to the DNA. This, in turn, effectively enhances gene transcription. On the other hand, deacetylation or removal of the acetyl group restores the positive charge condensing the structure of the nucleosome, thereby repressing gene expression. Thus, the balance between activities of histone acetylases, determines the level of histone acetylation.

Eighteen different HDACs have been cloned from vertebrate organisms. These proteins were grouped into three different classes based on sequence homologies and catalytic mechanism. Class I proteins share sequence homology with the yeast protein RPD3 and comprise HDACs 1, 2, 3 and 8. Class II proteins are homologous to the yeast HDA1 protein. HDACs 4, 5, 6, 7, 9, and 10 belong to this class. HDAC11 shares homology to both class I and class II enzymes. All class I and class II proteins, as well as HDAC11, are likely to make use of the same catalytic mechanism that involves the hydrolysis of an amide bond via a catalytic zinc ion. Class III enzymes, in contrast, which are homologous to the yeast Sir protein, differ from class I and class II proteins because they transfer an acetyl group from an acetylated lysine residue to NAD, simultaneously cleaving the dinucleotide. The molecular cloning of gene sequences encoding proteins with HDAC activity has established the existence of a set of discrete HDAC enzyme isoforms.

Recent research efforts have highlighted the important role of HDACs in cancer biology. Leukemic fusion proteins such as PML-RAR, PLZF-RAR or AML-ETO were shown to recruit HDACs inappropriately (Minucci et al. Molecular Cell 5, 811, 2000; Amann et al., Mol. Cell. Biol. 21, 6470, 2001). In B-cell lymphoma, aberrant expression of BCL6 was shown to lead to an anomalous recruitment of HDACs (Bereshchenko et al. Nat. Genet. 2002 December; 32(4):606-13.) In prostate and breast cancer, the severity of the disease was found to correlate with high expression levels of the polycomb protein EZH2, that recruits histone deacetylases and shows histone methyl transferase activities (Varambally et al., Nature 419, 624, 2002; Kleer et al., Proc. Natl. Acad. Sci. USA 100, 11606, 2003). Cancer is the second leading cause of human death next to coronary disease. Worldwide, millions of people die from cancer every year. In the United States alone, as reported by the American Cancer Society, cancer causes the death of well over a half-million people annually, with over 1.2 million new cases diagnosed per year.

Representative cancer types include carcinoma (e.g., adenocarcinoma), sarcoma, myeloma, leukemia, and lymphoma, and mixed types of cancers, such as adenosquamous carcinoma, mixed mesodermal tumor, carcinosarcoma, and teratocarcinoma. Exemplary cancers include, lung cancer, breast cancer, colon cancer, rectal cancer, endometrial cancer, and ovarian cancer as well as AIDS-related cancers (e.g., Kaposi's Sarcoma, AIDS-related lymphoma). Also included are brain cancers (e.g., adult brain tumor, childhood brain stem glioma, digestive/gastrointestinal cancers (e.g., colon cancer, esophageal cancer, gallbladder cancer, anal cancer, extrahepatic bile duct cancer, gastrointestinal carcinoid tumor). Also included are neurologic cancers (e.g., neuroblastoma, pituitary tumor, and primary central nervous system lymphoma), respiratory/thoracic cancers (e.g., non-small cell lung cancer, small cell lung cancer, malignant mesothelioma, and malignant thymoma), and gynecologic cancers (e.g., cervical cancer, endometrial cancer, gestational trophoblastic tumor, ovarian epithelial cancer, ovarian germ cell tumor, ovarian low malignant potential tumor, uterine sarcoma, vaginal cancer, and vulvar cancer), and unknown primary cancers.

HDAC inhibitors (HDACi) have been shown to inhibit tumor growth in animal models of breast, prostate, lung and stomach cancer, neuroblastoma and leukemia. These inhibitors have been found to have anti-proliferative effects, including induction of G1/S and G2/M cell cycle arrest, differentiation and apoptosis of transformed and normal cells and reversal of transformation. Arrest at G1/S is believed to be meditated by induction of the CDK-inhibitor p21. Support for this observation becomes evident when considering that a p21 defective colon cancer cell line failed undergo G1 arrest in response to HDACi. Several inhibitors are presently being evaluated as single agents and in combination regimens with cytotoxic agents for the treatment of advanced malignancies (reviewed in P. A. Marks et al., Curr. Opin. Oncol. 2001 November; 13(6):477-83).

Indeed, several small molecule inhibitors of HDAC have shown anti-proliferative activities on a number of tumor cell lines and potent anti-tumor activity in pre-clinical tumor xenograft models. Refer to CBHA (D. C. Coffey et al., 2001, Cancer Res. 61(9):3591-4), pyroxamide, (L. M. Butler et al, 2001, Clin. Cancer Res. 7(4): 962-70), and CHAP31 (Y. Komatsu et al., 2001, Cancer Res. 61(11):4459-66). For example, histone deacetylase inhibitors, phenylbutyrate and valproic acid have shown promise in the treatment of promyelocytic leukemia and several other HDAC inhibitors are being studied as treatments for cancers. Other potential HDAC inhibitors effective in arresting cell-proliferation include suberoylanilide hydroxamic acid (SAHA) (Butler et al., 2000; Marks et al., 2001); m-carboxycinnamic acid bis-hydroxamide (Coffey et al., 2001); and pyroxamide (Butler et al., 2001) as well as LAQ824 (Remiszewski, SW Curr. Med. Chem. 10, 2393, 2003), PXD101 (Plumb et al., Mol. Cancer. Ther. 2, 721, 2003), FK228 (Yoshida et al., Curr. Med. Chem. 10, 2351, 2003), MS27-275 (Saito et al., Proc. Natl. Acad. Sci. USA 96, 4592, 1999) and CI994 (Kraker et al., Mol. Cancer. Ther. 2, 401, 2003). The data suggest that inhibition of the action of HDACs causes a variety of cellular responses including the accumulation of hyperacetylated histones, altered gene expression, and cell cycle arrest.

Consequently, regulation of gene expression through the inhibition of the nuclear enzyme histone deacetylase (HDAC) thus represents one of several possible regulatory mechanisms whereby chromatin activity can be affected. As such, the inhibition of HDAC activity represents a novel approach for the intervention of cell cycle regulation and that HDAC inhibitors have great therapeutic potential in the treatment of cell proliferative diseases or conditions.

However, a distinct drawback attending the use of conventional HDAC inhibitors is the finding that all of the known histone deacetylase inhibitors are non-specific for a particular histone deacetylase isoform, and more or less inhibit all members of both the histone deacetylase families equally. See Grozinger, C. M., et al., Proc. Natl. Acad. Sci. U.S.A. 96:48684873, 1999. See also Marks et al., J. National Cancer Inst. 92:1210-1216 (2000), who review histone deacetylase inhibitors and their role in studying differentiation and apoptosis.

As well, there is a paucity of understanding of the pharmacology of HDACi molecules and their interaction with a target HDAC. Also, the identity of specific HDAC proteins and their association with specific anti-proliferative diseases is lacking. In addition, all known HDAC inhibitors have been shown to possess adverse effects such as mylosuppression and GI-toxicity, which, in turn, leads to dose-limitation, thereby adversely effecting their inhibitory action. This paucity of understanding, together with the limited knowledge in the art of the identification of specific HDAC subtypes and diseases associated therewith, has hampered the rational design, testing and screening of potent, selective HDAC inhibitors that interact with specific human HDAC subtypes. Thus, while a number of natural product and synthetic HDAC inhibitors have been reported (J. Med. Chem. 1999, 42, 3001; and PNAS, 1998, 95, 3003), there still exists a need for inhibitors with improved profiles of activity.

The instant application meets an unmet need by specifically identifying a specific HDAC protein as a potential target for inhibition as a means of treating specified cell proliferative disorders. As well, the invention provides for the development of methods for identifying HDAC modulators. An important feature of the invention is that not only does HDAC 11 knockdown strongly inhibit cell growth, it does so with less toxicity relative to other HDACi molecules.

### SUMMARY OF THE INVENTION

The present invention relates on the unexpected discovery of the role of HDAC 11 in certain cell proliferative disorders. The present inventors have established a nexus between HDAC 11 and certain cancers. The findings show that inhibitors of human HDAC 11 are useful in the treatment of certain cell proliferative disorders/cancers in that specific inhibition of HDAC 11 is sufficient to induce cell cycle arrest in said cell proliferative disorders notwithstanding that the expression of or activity of HDAC gene or its gene product in said cell proliferative disorders is normal or near normal.

Importantly, the surprising discovery forms the basis of the invention in that specific HDAC 11 inhibitors (HDAC 11i/HDAC Therapeutics) will aid in the treatment of specific cell proliferative disorders, i.e., colon cancer, lung cancer and cervical cancer.

Consequently, in a broad aspect, the invention relates to inhibition of human HDAC 11 as a means of treating certain cell proliferation disorders. The HDAC 11 inhibiting molecules may be biological entities such as dsRNA, antisense, or antibodies, etc or may be chemical moieties (small molecules) specific for HDAC 11.

In a first aspect, the invention provides a method for the treatment of a cell proliferative disorder, e.g., cancer, the method comprising administering to a subject in need thereof an agent which inhibits the bioactivity of human HDAC 11, or an agent which decreases expression of HDAC 11.

In a second aspect, the present invention provides a method for inhibiting the growth or proliferation of a tumor cell, the method comprising contacting a tumor cell with an agent which inhibits the activity of HDAC 11 or an agent which decreases expression of HDAC 11 in an amount sufficient in induce cell-cycle arrest.

In another aspect, the present invention provides a tumor/cancer cell into which a nucleic acid molecule has been introduced, the nucleic acid molecule comprising or encoding (i) an agent which decreases expression of HDAC 11 or (ii) an agent which inhibits HDAC 11 mediated activity.

In another aspect, the present invention provides a method of screening for an agent which inhibits cell proliferative disorders, the method comprising testing a putative agent for the ability to inhibit HDAC 11 bioactivity, or decrease expression of HDAC 11.

In a preferred embodiment of the present invention the agent is selected from the group consisting of a RNAi construct targeted for silencing HDAC 11 gene expression, an HDAC 11 antisense oligonucleotide, a ribozyme targeted against HDAC 11, an antibody specific for HDAC 11, a ssDNA targeted against HDAC 11dsDNA such that the ssDNA forms a triplex with the HDAC 11 dsDNA, an enzyme targeted against HDAC 11 and a chemical moiety (small molecule) that inhibits HDAC 11 function or activity.

The method of the first aspect may involve indirect inhibition of cancerous cell growth by inhibiting HDAC 11 mediated bioactivity or and/or direct inhibition by blocking HDAC 11 expression or activity in said cancer cells. As will be recognized by those skilled in this field there are a number of means by which the method of the present invention may be achieved.

In one embodiment, the method according to the first aspect proposes specifically inducing gene silencing, i.e., silencing the gene encoding HDAC 11 by a phenomenon commonly referred to as RNA interference.

In another embodiment, the method is achieved by targeting the HDAC 11 gene directly for example by using triple helix (triplex) methods in which a ssDNA molecule can bind to the dsDNA and prevent transcription.

In another embodiment, the method is achieved by inhibiting translation of the HDAC 11 mRNA using dsRNA constructs specific for a region of the HDAC 11 gene wherein the HDAC 11 gene expression is silenced via RNA interference.

In another embodiment, the method is achieved by inhibiting translation of the HDAC 11 mRNA using synthetic antisense DNA molecules that do not act as a substrate for RNase H and act by sterically blocking gene expression.

In another embodiment, the method is achieved by inhibiting translation of the HDAC 11 mRNA by destabilizing the mRNA using synthetic antisense DNA molecules that act by directing the RNase H-mediated degradation of the HDAC 11 mRNA present in the heteroduplex formed between the antisense DNA and mRNA.

In another embodiment, the method is achieved by inhibition of the function of a HDAC 11 gene product by drugs that effectively inhibit or block the deacetylation activity attendant a native HDAC 11 protein.

Within the context of the present invention, the HDAC 11 inhibitory agents may be administered either alone or in combination with one or more additional anti-cancer agents which will be known to a person skilled in the art.

Thus, an embodiment of the invention proposes using an RNAi construct that specifically downregulate expression of human HDAC 11, thereby effectively inducing cell-cycle arrest of cancerous cells. Thus, an aspect of the invention features small interfering nucleic acid molecule for modulating HDAC gene expression.

In accordance with the above, the invention provides methods of silencing a specific gene, via the use of a novel mechanism referred to herein as RNA interference (RNAi), which uses a specific RNAi construct effective to silence a specified gene. The method involves introducing a dsRNA specific for a region within a human HDAC 11 nucleotide sequence into a cell, and maintaining the cell under conditions and for a time sufficient to obtain degradation of mRNA of the target gene. The double-stranded structure comprises a nucleotide sequence which is substantially similar or identical to at least a part of a target gene in a mammalian cell. The RNA comprises a first complementary RNA strand and a second RNA strand, wherein the first complementary RNA strand comprises the nucleotide sequence corresponding to the target gene. Each of the first two RNA strands of the dsRNA have a 3′-terminus and a 5′-terminus. The siRNA comprises between about 20 and about 24 nucleotides in length, preferably 22 nucleotides (siRNA construct), and the target gene comprises a contiguous sequence of nucleotides within the target gene, e.g., human HDAC gene.

At least one of the two RNA strands of the dsRNA (siRNA construct) may have a nucleotide overhang of between one and four nucleotides, preferably one or two nucleotides, in length. The dsRNA may have only one nucleotide overhang, preferably on the 3′-terminus of the complementary RNA strand. At least one of the ends of the dsRNA may further comprise a chemical linker, such as a hexaethylene glycol linker. The linker may connect the 5′-terminus of the first complementary RNA strand and the 3′-terminus of the second RNA strand.

The first RNA strand of the dsRNA may have the nucleotide sequence of any one of SEQ ID NOS: 3-6; and the second RNA strand may have a complementary nucleotide sequence. Each of the first strand has a sequence of nucleotides substantially identical/complementary to a portion of the sequence of nucleotides set forth in SEQ ID NO: 1. The dsRNA may comprise a single-self complementary RNA strand, wherein one end comprises a loop structure and the other end comprises the two termini. The dsRNA may have a nucleotide overhang of between about one and about four nucleotides, preferably one or two nucleotides, in length. The small interfering nucleic acid molecule of the invention can be unmodified or chemically-modified.

In a further aspect, the invention relates to a pharmaceutical composition comprising the dsRNA and a pharmaceutically acceptable carrier. The mammalian cell may be a neoplastic cell or other cancerous cell. The target gene is human HDAC 11.

In another embodiment, the invention features dsRNA constructs comprising a sequence selected from the group consisting of SEQ ID NOS: 3-10.

An alternative embodiment of the present invention is drawn to a purified or isolated antisense nucleic acid comprising a nucleic acid sequence complementary to at least a portion of a target gene effective to silence said gene. The portion may be an intragenic sequence, intergenic sequence, sequences spanning at least a portion of two or more genes, 5′ noncoding region, or 3′ noncoding region within an operon comprising a proliferation-required gene whose activity or expression is inhibited by an antisense nucleic acid. The target gene is human HDAC 11 of SEQ ID NO: 1 including variants and biologically active fragments thereof.

Another embodiment of the present invention is a vector comprising a promoter operably linked to a nucleic acid encoding a polypeptide whose expression is inhibited by an antisense nucleic acid comprising a nucleic acid sequence complementary to at least a portion of a target gene effective to silence said gene. Preferably, the target gene is HDAC 11. Another embodiment of the present invention is a method of inhibiting cell proliferative disorders comprising inhibiting the activity or reducing the amount of a gene product whose expression is inhibited by an antisense nucleic acid as noted above. The gene product may comprise a polypeptide comprising a sequence as set forth in SEQ ID NO:2. Variants and fragments of said polypeptide are included.

In an exemplary embodiment, an antisense method is used to treat tumor cells by antagonizing HDAC activity and blocking cell cycle progression. The method includes, but is not limited to, the treatment of lung tissue, cervical tissue or colon cell so as to modulate HDAC 11 expression or activity with a biological or chemical moiety effective to inhibit said HDAC 11 activity or expression.

In certain embodiments, the biological moiety may be an antibody specific for the gene product of SEQ ID NO: 1 effective to antagonize HDAC 11 mediated activity. In other embodiments the HDACi moiety may be a chemical entity capable of antagonizing the histone deacetylase activity of HDAC 11 effective to induce cell-cycle arrest in said cells contacted with said chemical entity.

Another aspect of the invention is directed to methods for the identification of molecules that can bind to the gene product of SEQ ID NO: 1 or variants there such as to inhibit activity of said gene product, e.g., deacetylase activity attendant a native HDAC enzyme.

In accordance with the above, the invention provides methods for the identification of molecules that can bind to a polypeptide comprising an amino acid sequence set forth in SEQ ID NO: 2 and or modulate the activity of a polypeptide comprising an amino acid sequence set forth in SEQ ID NO: 2 or molecules that can bind to nucleic acid sequences that modulate the transcription or translation of a polynucleotide encoding a polypeptide comprising an amino acid sequence set forth in SEQ ID NO: 2.

Another aspect of the present invention involves a method for modulating HDAC bioactivity, e.g., by inhibiting the deacetylase activity of HDAC proteins, or disrupting certain protein-protein interactions. In general, whether carried out in vivo, in vitro, ex vivo, or in situ, the method comprises treating a cell with an effective amount of an HDAC therapeutic so as to alter, relative to an effect in the absence of treatment, one or more of (i) rate of growth or proliferation, (ii) differentiation, or (iii) survival of the cell. Accordingly, the method can be carried out with HDAC therapeutics, such as peptide and peptidomimetics, or other molecules identified in the drug screening methods as described herein which antagonize the effects of a naturally-occurring HDAC protein on a cell.

Yet another aspect of the invention relates to compounds and compositions useful for modulating mammalian HDAC 11 function and/or gene expression in a cell.

Molecules identified by such methods also fall within the scope of the present invention.

The proposed nucleic acid molecules of the instant invention, e.g., siRNA and antisense constructs, antibodies, peptides etc. together with chemical moieties, e.g., small molecules identified using the methods of the invention provide useful reagents and methods for a variety of therapeutic applications including in particular the treatment of cell proliferative disorders such as cancers of the cervix, lung and colon.

### BRIEF DESCRIPTION OF THE DRAWINGS

FIG. 1 details the gene knock down of HDAC11 in human cells. The upper panel details the results of a western blot analysis of HCT116 cells transfected with 4 different siRNA constructs, e.g., 11.2, 11.3, 11.4, 11.5 each of which is directed against a specified region within the HDAC11 mRNA 48 hours post-transfection.

FIGS. 2a and 2b detail the cell growth curves of HCT116 or A549 cells transfected with siRNA constructs of the invention directed against HDAC11 or GL2 (Control).

FIG. 3 represents the nucleotide sequence of Human HDAC 11 (SEQ ID NO: 1) according to an embodiment of the invention.

FIG. 4 shows the deduced amino acid sequence of the HDAC 11 polypeptide of the invention (SEQ ID NO: 2).

### DETAILED DESCRIPTION OF THE INVENTION

The description below of the various aspects and embodiments is provided with reference to the exemplary human HDAC 11 protein. However, the various aspects and embodiments are also directed to other genes that are members of the HDAC family, particularly those that exhibit similar biological functions.

Before the present proteins, nucleotide sequences, and methods are described, it is to be understood that the present invention is not limited to the particular methodologies, protocols, cell lines, vectors, and reagents described, as these may vary. It is also understood that the terminology used herein is for the purpose of describing particular embodiments only, and is not to limit the scope of the present invention.

### GLOSSARY

The singular forms “a,” “an,” and “the” include plural reference unless the context clearly dictates otherwise.

All technical and scientific terms used herein have the same meanings as commonly understood by one of ordinary skill in the art to which this invention pertains. The practice of the present invention will employ, unless otherwise indicated, conventional techniques of protein chemistry and biochemistry, molecular biology, microbiology and recombinant DNA technology, which are within the skill of the art. Such techniques are explained fully in the literature. All patents, patent applications, and publications mentioned herein, whether supra or infra, are each incorporated by reference in its entirety.

For convenience, the meaning of certain terms and phrases employed in the specification, examples, and appended claims are provided below. It is also to be understood that the terminology used herein is for the purpose of describing particular embodiments only and is not intended to be limiting.

By “gene” oligonucleotide nucleic acid as used herein, refers to an oligonucleotide, nucleotide, or polynucleotide (e.g., DNA, cDNA, RNA), and fragments or portions thereof, and to DNA or RNA of genomic or synthetic origin which may be single- or double-stranded, and represent the sense (coding) or antisense (non-coding) strand. The exact size will depend on many factors, which in turn depends on the ultimate function or use of the oligonucleotide. The oligonucleotide may be generated in any manner, including chemical synthesis, DNA replication, reverse transcription. The term should also be understood to include, as equivalents, analogs of either RNA or DNA made from nucleotide analogs, and, as applicable to the embodiment being described, single (sense or antisense) and double-stranded polynucleotides. As a non-limiting example, fragments include nucleic acid sequences that can be about 10 to 60 contiguous nucleotides in length, preferably, at least 15-60 contiguous nucleotides in length, and also preferably include fragments that are at least 70-100 contiguous nucleotides, or which are at least 1000 contiguous nucleotides or greater in length. Nucleic acids for use as probes or primers may differ in length as described herein.

As used herein, “target gene” refers to a section of a DNA strand of a double-stranded DNA that is complementary to a section of a DNA strand, including all transcribed regions, that serves as a matrix for transcription. The target gene is therefore usually the sense strand. The target gene can be a gene derived from a cell, an endogenous gene, a transgene, or exogenous genes such as genes of a pathogen, for example a virus, which is present in the cell after infection thereof. The cell containing the target gene can be derived from or contained in any organism, for example a plant, animal, protozoan, virus, bacterium, or fungus. Non-limiting examples of animals include vertebrates or invertebrates. Non-limiting examples of fungi include molds or yeasts. Preferably, the target gene is human HDAC 11 including variants and biologically equivalent sequences thereof.

When referring to a sequence that “consists of about” a certain number of nucleotides, this is intended to refer to a sequence that consists of the certain number of nucleotides plus or minus 20% or 10% of the number of nucleotides. For example, a sequence consisting of about 10 nucleotides refers to a sequence of from 8-12 nucleotides.

A “delivery complex” or “siRNA vehicle” shall mean a targeting means for delivering an siRNA complex to a target gene. Examples of targeting means include: sterols (e.g. cholesterol), lipids (e.g. a cationic lipid, virosome or liposome), viruses (e.g. adenovirus, adeno-associated virus, and retrovirus) or target cell specific binding agents (e.g. ligands recognized by target cell specific receptors).

The term “equivalent” is understood to include nucleotide sequences encoding functionally equivalent polypeptides. Equivalent nucleotide sequences will include sequences that differ by one or more nucleotide substitutions, additions or deletions, such as allelic variants; and will, therefore, include sequences that differ from the nucleotide sequence of a nucleic acid of interest due to the degeneracy of the genetic code. Thus “equivalent” nucleotide sequences of human HDAC of SEQ ID NO: 1 include degenerate sequences.

“Identity,” as known in the art, is the relationship between two or more polynucleotide sequences, as determined by comparing the sequences. Identity also means the degree of sequence relatedness between polynucleotide sequences, as determined by the match between strings of such sequences. Identity can be readily calculated (see, e.g, Computation Molecular Biology, Lesk, A. M., eds., Oxford University Press, New York (1998), and Biocomputing: Informatics and Genome Projects, Smith, D. W., ed., Academic Press, New York (1993), both of which are incorporated by reference herein). While there exist a number of methods to measure identity between two polynucleotide sequences, the term is well known to skilled artisans (see, e.g., Sequence Analysis in Molecular Biology, von Heinje, G., Academic Press (1987); and Sequence Analysis Primer, Gribskov, M. and Devereux, J., eds., M Stockton Press, New York (1991)). Methods commonly employed to determine identity between sequences include, for example, those disclosed in Carillo, H., and Lipman, D., SIAM J. Applied Math. (1988) 48:1073. “Substantially identical,” as used herein, means there is a very high degree of homology (preferably 100% sequence identity) between the respective target sequence and the reference sequence. Thus, where an embodiment is drawn to silencing a particular gene via t RNA interference, “substantially identical” refers to the nucleotide sequence comprising the strand of the dsRNA which is complementary to an mRNA of the target gene or a region contained therein. The percentage of identity between the substantially similar nucleotide sequences is at least 80%, more desirably at least 85%. However, dsRNA having greater than 90%, or 95% sequence identity may be used in the present invention, and thus sequence variations that might be expected due to genetic mutation, strain polymorphism, or evolutionary divergence can be tolerated. Although 100% identity is preferred, the dsRNA may contain single or multiple base-pair random mismatches between the RNA and the target gene.

The term “complementary RNA strand” refers to the strand of the dsRNA which is complementary to an mRNA transcript that is formed during expression of the target gene, or its processing products. “dsRNA” refers to a ribonucleic acid molecule having a duplex structure comprising two complementary and anti-parallel nucleic acid strands. Not all nucleotides of a dsRNA must exhibit Watson-Crick base pairs. The maximum number of base pairs is the number of nucleotides in the shortest strand of the dsRNA.

As an example, a nucleotide sequence when referring to an anti-sense construct (reference sequence) is “substantially similar” to the target nucleotide sequence when said sequence hybridizes to the reference nucleotide sequence in 7% sodium dodecyl sulfate (SDS), 0.5 M NaPO4, 1 mM EDTA at 50° C. with washing in 2×SSC, 0.1% SDS at 50° C., more desirably in 7% sodium dodecyl sulfate (SDS), 0.5 M NaPO4, 1 mM EDTA at 50° C. with washing in 1×SSC, 0.1% SDS at 50° C., more desirably still in 7% sodium dodecyl sulfate (SDS), 0.5 M NaPO4, 1 mM EDTA at 50° C. with washing in 0.5×SSC, 0.1% SDS at 50-C, preferably in 7% sodium dodecyl sulfate (SDS), 0.5 M NaPO4, 1 mM EDTA at 50° C. with washing in 0.1×SSC, 0.1% SDS at 50° C., more preferably in 7% sodium dodecyl sulfate (SDS), 0.5 M NaPO4, 1 mM EDTA at 50° C. with washing in 0.1×SSC, 0.1% SDS at 65° C., yet still encodes a functionally equivalent gene product.

An allele or allelic sequence is an alternative form of an HDAC nucleic acid sequence. Alleles may result from at least one mutation in the nucleic acid sequence and may yield altered mRNAs or polypeptides whose structure or function may or may not be altered. Any given gene, whether natural or recombinant, may have none, one, or many allelic forms. Common mutational changes that give rise to alleles are generally ascribed to natural deletions, additions, or substitutions of nucleotides. Each of these types of changes may occur alone, or in combination with the others, one or more times in a given sequence.

“RNAi” stands for RNA-mediated interference.

The term “short interfering RNA” or “siRNA” as used herein refers to a double stranded nucleic acid molecule capable of RNA interference “RNAi”, see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. As used herein, siRNA molecules need not be limited to those molecules containing only RNA, but further encompasses chemically modified nucleotides and non-nucleotides.

“A therapeutically effective amount” of a compound is an amount which results in a therapeutic effect in the subject to whom it was administered.

“Inhibiting gene expression” “gene silencing” refers to a phenomenon whereby a function of a gene is completely or partially inhibited. For example, the action may result in decreased production of a polypeptide encoded by the gene or decreased levels of an RNA encoded by the target gene. Inhibiting gene expression includes inhibiting transcription, translation or degrading the DNA template or RNA encoded thereby. As such, HDACi molecules may be characterized as having the ability to interfere with the function of a gene or gene product in such a way as to decrease expression of the gene or to reduce the level or activity of a product of the gene. Agents which inhibit the activity of a gene include agents that inhibit transcription of the gene, agents that inhibit processing of the transcript of the gene, agents that reduce the stability of the transcript of the gene, and agents that inhibit translation of the mRNA transcribed from the gene. Of particular utility to the present invention are dsRNA molecules, antisense RNAs that have activities against the operons or genes to which they specifically hybridize. For example, inhibiting histone deacetylation causes cells to arrest in the G1 and G2 phases of the cell cycle. In general, whether the HDAC 11i molecule is an antisense or an siRNA molecule, inhibition of gene expression with the siRNA molecule or an anti-sense molecule is greater in the presence of the siRNA or anti-sense molecule than in its absence.

According to an aspect of the invention, there is provided a HDAC therapeutic which causes an inhibition of cell proliferation of the contacted cells. The phrase “inhibiting cell proliferation” is used to denote an ability of an inhibitor of histone deacetylase to retard the growth of cells contacted with the inhibitor as compared to cells not contacted. An assessment of cell proliferation can be made by counting contacted and non-contacted cells using a Coulter Cell Counter (Coulter, Miami, Fla.) or a hemocytometer. Where the cells are in a solid growth (e.g., a solid tumor or organ), such an assessment of cell proliferation can be made by measuring the growth with calipers and comparing the size of the growth of contacted cells with non-contacted cells.

Preferably, growth of cells contacted with the inhibitor is retarded by at least 50% as compared to growth of non-contacted cells. More preferably, cell proliferation is inhibited by 100% (i.e., the contacted cells do not increase in number). Most preferably, the phrase “inhibiting cell proliferation” includes a reduction in the number or size of contacted cells, as compared to non-contacted cells. Thus, an inhibitor of historic deacetylase according to the invention that inhibits cell proliferation in a contacted cell may induce the contacted cell to undergo growth retardation, to undergo growth arrest, to undergo programmed cell death (i.e., to apoptosis), or to undergo necrotic cell death.

In some preferred embodiments, the contacted cell is a neoplastic cell. The term “neoplastic cell” is used to denote a cell that shows aberrant cell growth. Preferably, the aberrant cell growth of a neoplastic cell is increased cell growth. A neoplastic cell may be a hyperplastic cell, a cell that shows a lack of Contact inhibition of growth in vitro, a benign tumor cell that is incapable of metastasis in vivo, or a cancer cell that is capable of metastasis in vivo and that may recur after attempted removal. The term “tumorigenesis” is used to denote the induction of cell proliferation that leads to the development of a neoplastic growth.

In some preferred embodiments, the contacted cell is in an animal. Thus, the invention provides a method fox treating a cell proliferative disease or condition in an animal, comprising administering to an animal in need of such treatment a therapeutically effective amount of a histone deacetylase inhibitor of the invention. Preferably, the animal is a mammal, more preferably a domesticated mammal. Most preferably, the animal is a human. Preferably, the histone deacetylase is HDAC 11.

The term “cell proliferative disease or condition” is meant to refer to any condition characterized by aberrant cell growth, preferably abnormally increased cellular proliferation. In particularly preferred embodiments, the invention provides a method for inhibiting neoplastic cell proliferation in an animal comprising administering to an animal having at least one neoplastic cell present in its body a therapeutically effective amount of a histone deacetylase inhibitor of the invention.

As used herein, the terms “histone deacetylase” and “HDAC” are intended to refer to any one of a family of enzymes that remove acetyl groups from the Σ-amino groups of lysine residues at the N-terminus of a histone. Unless otherwise indicated by context, the term “histone” is meant to refer to any histone protein, including H1, H2A, H2B, H3, H4, and H5, from any species. Preferably the histone deacetylase is a human HDAC, and most preferably the human histone deacetylase is human HDAC 11. The HDAC 11 of the invention is characterized as proteins capable of removing acetyl groups from primary amines or amino acids, either free or in the context of a polypeptide chain.

The term “HDACi” (histone deacetylase inhibitor) refers to any agent that inhibits HDAC 11 activity or expression of the HDAC 11 gene. Where the HDACi is a small molecule, it is capable of inhibiting HDAC activity, by for example, interacting with a histone deacetylase and inhibiting its enzymatic activity. Inhibiting histone deacetylase enzymatic activity means reducing the ability of a histone deacetylase to remove an acetyl group from a histone. In some preferred embodiments, such reduction of histone deacetylase activity is at least about 50%, more preferably at least about 75%, and still more preferably at least about 90%. In other preferred embodiments, histone deacetylase activity is reduced by at least 95% and more preferably by at least 99%.

An “HDAC therapeutic,” whether inhibitory or potentiating with respect to modulating histone deacetylation, can be, as appropriate, any of the preparations described herein, including isolated polypeptides, gene therapy constructs, antisense molecules, or agents identified in the drug and bioactive screening assays and methods well known to one skilled in the art as well as those described herein.

The term “sample” or “biological sample”, is meant to be interpreted in its broadest sense. A biological sample suspected of containing nucleic acid encoding an HDAC protein, or fragments thereof, or an HDAC protein itself, may comprise a body fluid, an extract from cells or tissue, chromosomes isolated from a cell (e.g., a spread of metaphase chromosomes), organelle, or membrane isolated from a cell, a cell, nucleic acid such as genomic DNA (in solution or bound to a solid support such as for Southern analysis), RNA (in solution or bound to a solid support such as for Northern analysis), cDNA (in solution or bound to a solid support), a tissue, a tissue print and the like. Methods for detecting HDAC 11 expression are well known, e.g., Northern Blot, Southern Blot, Western Blot etc.

An HDAC-11 protein refers to the HDAC 11 proteins or polypeptides described herein, as well as other human homologs of these HDAC sequences, in addition to orthologs and paralogs (homologs) of the HDAC sequences in other species, ranging from yeast to other mammals, e.g., homologous histone deacetylase. The full-length cDNA for HDAC 11 is as set forth in SEQ ID NO: 1 and it predicts a protein comprising the amino acid sequence as set forth in SEQ ID NO:2. The term ortholog refers to genes or proteins that are homologs via speciation, e.g., closely related and assumed to have common descent based on structural and functional considerations. Orthologous proteins function as recognizably the same activity in different species. The term paralog refers to genes or proteins that are homologs via gene duplication, e.g., duplicated variants of a gene within a genome. (See, W. M. Fritch, 1970, Syst. Zool., 19:99-113.

It will be appreciated that, under certain circumstances, it may be advantageous to provide homologs of the preferred HDAC polypeptides which function in a limited capacity as one of either an HDAC agonist (i.e., mimetic), or an HDAC antagonist, in order to promote or inhibit only a subset of the biological activities of the naturally-occurring form of the protein. Thus, specific biological effects can be elicited by treatment with a homolog of limited function, and with fewer side effects, relative to treatment with agonists or antagonists which are directed to all of the biological activities of naturally-occurring forms of HDAC proteins. Homologs (i.e., isoforms or variants) of a target HDAC polypeptides can be generated by mutagenesis, such as by discrete point mutation(s), or by truncation. For example, mutation can yield homologs that retain substantially the same, or merely a subset of, the biological activity of the HDAC polypeptide from which it was derived. Alternatively, antagonistic forms of the protein can be generated which are able to inhibit the function of the naturally-occurring form of the protein, such as by competitively binding to an HDAC substrate, or HDAC-associated protein. Non-limiting examples of such situations include competing with wild-type HDAC in the binding of p53 or a histone. Also, agonistic forms of the protein can be generated which are constitutively active, or have an altered K_{cat} or Kₘ for deacylation reactions. Thus, the HDAC protein and homologs thereof may be either positive or negative regulators of transcription and/or replication.

As used herein, “HDAC activity”, including refers to the ability of an HDAC polypeptide to deacetylate histone proteins.

The term “biologically active”, i.e., functional, refers to a protein or polypeptide or peptide fragment thereof having structural, regulatory, or biochemical functions of a naturally occurring molecule. Likewise, “immunologically active” refers to the capability of the natural, recombinant, or synthetic HDAC, or any oligopeptide thereof, to induce a specific immune response in appropriate animals or cells, for example, to generate antibodies, and to bind with specific antibodies.

In specific embodiments, a polynucleotide of the invention can comprise a sequence of nucleotides that specifically target a region of an HDAC 11 nucleotide sequence via RNA interference. Said regions may include nucleotides positions 511-531, 580-600, 1030-1050 and 1342-1362 of the native HDAC gene sequence, represented herein as SEQ ID NO: 1.

### Methods of the Invention

Several human cancers have been associated with malfunctions in HAT and HDAC activity. Indeed, it is well documented that multiple classical features of cancer cells can be manifested by improper histone deacetylation. For review see Wade, P. A. Hum Mol tenet; 10(7):693-698 (2001. In addition, perturbations of gene expression have long been acknowledged to account for a vast number of diseases including, numerous forms of cancer, vascular diseases, neuronal and endocrine diseases. Consequently, one of the major challenges of medicine has been to regulate the expression of targeted genes that are implicated in a wide diversity of physiological responses.

However, conventional treatments for cancer, are marred by major drawbacks. For example, current treatment protocols for cell proliferative disorders, such as cancer require empirically derived cytotoxic chemotherapy that is marginally more toxic for the hyper proliferative cell than normal cells.

The therapeutic effects of HDAC inhibition are believed to occur through the induction of differentiation and/or apoptosis through the up-regulation of genes such as the cyclin dependent kinase inhibitors, p21 and p27 (see, e.g., W. Wharton et al., 2000, J. Biol. Chem. 275(43):33981-7; L. Huang et al., 2000, Mol. Med. 6(10):849-66). However, although known HDAC inhibitors are efficacious as anti-tumor agents, they are also associated with toxicity (see, e.g., V. Sandor et al., 2002, Clin. Cancer Res. 8(3):718-28). Such toxicity is believed to be caused by a non-selective mechanism of targeting multiple HDACs. Thus, despite the potent anti-tumor activity of HDAC inhibitors, it is still unclear which HDACs are necessary to produce an anti-proliferative response. Furthermore, little progress has been made in comparing the HDAC gene expression profiles in tumor versus normal cells. Differential HDAC expression may underlie the tumor-selective responses of HDAC inhibition. In addition, a cellular growth advantage may be conferred by the expression of particular HDAC's.

As regards gene inhibition as an alternative strategy for treating cancer, targeted inhibition of specific genes has been very difficult to achieve. As well, current approaches for suppressing gene expression, including site-directed gene disruption etc require complex genetic manipulations or heavy dosages of suppressors that often exceeds the toxicity tolerance level of the host cell. Therefore, there is a need for further insight into the consequences of selective HDAC inhibition, or activation.

Thus, while conventional methods for either the prevention or the alleviation of symptoms and/or the slowing down of disease progression may improve quality of life in cancer patients, there exists an unmet need in the comprehensive treatment and prevention of said cancerous disorders, especially those treatments where specific enzymes are targeted, thereby improving the overall treatment protocol. As such, there exists the need for therapeutics effective not only in reversing the physiological changes associated with hyper cell proliferative disorders but also being more selective and potent with fewer toxic side effects. The use of compounds to modulate the expression of selective HDAC subtypes, e.g., HDAC 11 is of therapeutic significance.

With respect to therapeutic intervention via agents which target expression of genes, these generally require acute specificity and/or are time consuming. For example, zinc-finger proteins (Choo, Y., et al., Nature (1994) 372:642), act at the DNA level, interacting with the target sequence and blocking transcription. However, gene fusions occur randomly and within introns, hence requiring a unique or “custom” zinc-finger for each patient. Hammerhead ribozymes, (James, H. A, and 1. Gibson, Blood (1998) 91:371) in turn, require specific nucleotide sequences in the target genie, which are not always present.

More recently, double-stranded RNA molecules (dsRNA) have been shown to block gene expression in a highly conserved regulatory mechanism known as RNA interference (RNAi). The presence of long dsRNAs in cells is believed to stimulate the activity of a ribonuclease III enzyme referred to as dicer. Dicer is involved in the processing of the dsRNA into short pieces of dsRNA known as short interfering RNAs (siRNA) (Berstein et al., 2001, Nature, 409, 363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188). In other words, RNAi involves a catalytic-type reaction whereby new siRNAs are generated through successive cleavage of long dsRNA. Thus, unlike antisense, RNAi degrades target RNA in a non-stoichiometric manner.

Initial attempts to harness this phenomenon for experimental manipulation of mammalian cells were foiled by a robust and nonspecific antiviral defense mechanism activated in response to long dsRNA molecules. Gil et al. Apoptosis 2000, 5:107-114. The field was significantly advanced upon the demonstration that synthetic duplexes of 21 nucleotide RNAs when administered to a cell or organism, provoke degradation of endogenous messenger RNA (mRNA) through RNAi., without involving a generic antiviral defense mechanisms. Elbashir et al. Nature 2001, 411:494-498; Caplen et al. Proc Natl Acad Sci 2001, 98:9742-9747. As a result, small-interfering RNAs (siRNAs) have become powerful tools to dissect gene function. Numerous groups have sought the development of DNA-based vectors capable of generating such siRNA within cells.

Several groups have recently attained this goal and published similar strategies that, in general, involve transcription of short hairpin (sh)RNAs that are efficiently processed to form siRNAs within cells. Paddison et al. PNAS 2002, 99:1443-1448; Paddison et al. Genes & Dev 2002, 16:948-958; Sui et al. PNAS 2002, 8:5515-5520; and Brummelkamp et al. Science 2002, 296:550-553. These reports describe methods to generate siRNAs capable of specifically targeting numerous endogenously and exogenously expressed genes. In addition, dsRNA has been reported to have anti-proliferative properties, which makes it possible also to envisage therapeutic applications (Aubel et al., Proc. Natl. Acad. Sci., USA 88:906 (1991)). For example, synthetic dsRNA has been shown to inhibit tumor growth in mice (Levy et al. Proc. Nat. Acad. Sci. USA, 62:357-361 (1969)), is active in the treatment of leukemic mice (Zeleznick et al., Proc. Soc. Exp. Biol. Med. 130:126-128 (1969)); and inhibits chemically-induced tumorigenesis in mouse skin (Gelboin et al., Science 167:205-207 (1970)).

Consequently, the invention provides a reliable method for inhibiting the proliferation and migration of tumor cells in human patients, and for inhibiting metastatic cancer development. It is believed that such a method would have implications for the therapeutic treatment of cell proliferative disorder sin general, and in particular those disorders that are mediated by HDAC 11. In the main, the invention relies on the discovery that various hyper proliferative disorders such as some forms of cancer may be amenable to treatment by specifically inhibiting the expression or biological activity of HDAC 11. The details of these experiments are outlined in the Examples herebelow.

In its broadest aspect, the invention provides HDAC modulatory (i.e., preferably inhibitory) compounds e.g., HDACi molecules that are likely to play an important role in treating or reducing cellular proliferation. Preferred histone deacetylase inhibitors are those that interact with and reduce the enzymatic activity of a histone deacetylase that is involved in tumorigenesis. Specifically, the HDAC modulating moieties can be chemical moieties capable of inhibiting HDAC 11 activity in vivo or ex vivo or biological moieties capable of inhibiting HDAC 11 activity or expression levels in vivo. Exemplary HDAC modulating moieties that are biological in nature include siRNA duplexes capable of specifically silencing HDAC expression in vivo or anti-sense constructs capable of specifically inhibiting HDAC 11 gene transcription or translation in vivo or antibodies or peptides that inhibit HDAC 11 protein function, some of which are exemplified in greater detail here after.

In certain aspects of the inventions, the disclosed HDAC 11 inhibitors, antisense molecules, anti-HDAC antibodies, or antibody fragments can be used as treatments for colon, cervical or lung cancer.

### Inhibition of HDAC 11 Expression

### (I) RNAi Based Embodiments

In one approach, the present invention relates to the specific inhibition of expression of a histone deacetylase gene in a mammal using a short double stranded RNA (dsRNA). As noted, supra, dsRNA directs the sequence-specific degradation of mRNA through a process known as RNA interference (RNAi). As detailed in the Examples section, the present inventors have demonstrated that dsRNA of approximately 19-24 nucleotides, preferably 20-23 nucleotides, and most preferably 22 nucleotides in length, which have a nucleotide sequence complementary to the target gene or to a region contained therein, can specifically and efficiently mediate RNAi. The present invention encompasses these short dsRNAs and their use for specifically inactivating gene function. The use of these dsRNAs enables the targeting of mRNAs of mammalian gene involved in cell proliferative disorders. Thus, the dsRNAs of the present invention are useful for treating diseases caused by a specified HDAC gene, particularly malignant diseases such as lung cancer, cervical cancer and cancer of the colon.

The dsRNAs of the present invention comprises a double stranded structure, and have a nucleotide sequence which is substantially identical to at least a part or portion of the target gene. Preferably, the target gene is HDAC 11 including fragments and biologically equivalent variants thereof.

Consequently, the discovery of a nexus between HDAC 11 and certain cancerous conditions, the siRNA molecules of the invention together with the methods detailed herein represent a novel therapeutic approach to treat a variety of pathologic indications, particularly cancer and any other diseases or conditions that are related to the levels of human HDAC level of expression or activity in a cell or tissue, alone or in combination with other therapies. The reduction of HDAC activity or expression and thus reduction in the level of the respective protein relieves, to some extent, the symptoms of the disease or condition.

Preferably, each sequence of a siRNA molecule of the invention is independently about 11 to about 24 nucleotides in length, more preferably of from about 17 to about 23, e.g, about 17, 18, 19, 20, 21, 22, or 23 base pairs. In some instances, the length of the nucleotide sequence is such as to be effective in silencing the target gene.

In accordance with an embodiment of the invention, the invention relates to an RNA having a double-stranded structure and a nucleotide sequence which is substantially identical to at least a part of the target gene. The RNA is between about 19 and about 24 nucleotides in length. The dsRNA comprises two complementary RNA strands, one of which comprises a nucleotide sequence which is substantially identical to a portion of the target gene. In a preferred embodiment, at least one end of the dsRNA has a single-stranded nucleotide overhang of between one and four, preferably one or two nucleotides. As used herein, a “nucleotide overhang” refers to the unpaired nucleotide or nucleotides that protrude from the duplex structure when the 5′-terminal end of one RNA strand extends beyond the 3′-terminus end of the other strand, or vice versa dsRNAs having at least one nucleotide overhang have unexpectedly superior inhibitory properties than their blunt-ended counterparts. It is well accepted that the presence of one or two nucleotide overhang appears to strengthen the interference activity of the respective dsRNA, without diminishing the overall stability of the structure, as typically happens with other dsRNA having no overhang. Preferably, the single-stranded overhang is located at the 3′-terminal end of the complementary RNA strand (also referred to herein as the “S1” strand). It is believed that such a configuration produces a further increase in efficiency.

The nucleotide sequence on the complementary RNA strand (S1 strand) preferably has between 18 and 24 nucleotides, most preferably 19-21 nucleotide s. The complementary RNA strand of the dsRNA preferably has fewer that 23 nucleotides because as noted by researchers in the field of RNAi such dsRNA molecules exhibit superior intracellular stability and avoid an interferon response.

At least one end of the dsRNA may be modified to improve resistance to degradation and/or dissociation of the two strands of the duplex. As well, the cohesiveness of the double-stranded structure formed by base pairing between the complementary RNA strands can be further improved by the presence of one, and preferably two, chemical linkages. Chemical linking may be achieved by any of a variety of well-known techniques, including through covalent, ionic or hydrogen bonds; hydrophobic interactions, preferably van der Waals or stacking interactions; or by means of metal-ion coordination. The purines of the dsRNA may also be replaced with purine analogues. Most preferably, the chemical linkage is achieved using a hexa-ethylene glycol linker on one end of the dsRNA. In a preferred embodiment, the linkage is formed between the 5′-terminus of the complementary RNA strand and the 3′-terminus of the second RNA strand.

In another embodiment, the present invention relates to a method for inhibiting the expression of a target gene comprising a fusion site using a dsRNA. The method comprises introducing a dsRNA having a nucleotide sequence which is substantially identical to at least a part of a target gene into a mammalian cell. The RNA is preferably between 20 and 23 nucleotides in length, most preferably 22 nucleotides. The resulting cell is maintained under conditions and for a time sufficient to achieve degradation of mRNA of the target gene, thereby silencing expression of the target gene.

In still another embodiment, the invention relates to a method for treating a mammal having a disease caused by the expression of a mammalian HDAC, preferably HDAC 11. The method comprises administering the dsRNA of the invention to the animal, such that expression of the target gene is silenced.

In an exemplified embodiment, the target gene comprises a HDAC gene, i.e., HDAC 11 gene. In this example, the complementary RNA (S1) strand of the dsRNA has the sequence selected from the groups consisting of SEQ ID NOS: 3-6, and the second (S2) strand has the sequence of SEQ ID NO:7-10. Such a construct is useful for treating either colon, cervical or lung cancer. The dsRNA (siRNA molecule) may be administered using any acceptable method known to one skilled in the art, including liposomes etc. Where the HDAC 11i is a siRNA molecule and its use is envisioned as a therapeutic, such therapeutic agents can be administered by a variety of well known techniques, including inhalation, oral ingestion, and injection, particularly intravenous or intraperitoneal injection, or injection directly into the affected bone marrow.

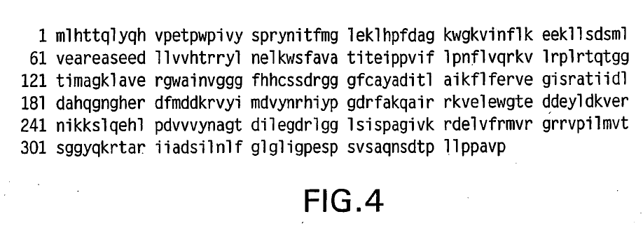

The siRNA molecules of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In particular embodiments, the nucleic acid molecules of the invention comprise sequences shown in Table I and/or FIGS. 4 and 5. Examples of such nucleic acid molecules consist essentially of sequences defined in this table.

In another aspect, the invention provides mammalian cells containing one or more siRNA molecules of this invention. The one or more siRNA molecules can independently be targeted to the same or different sites.

The nucleic acid molecules of the instant invention, individually, or in combination or in conjunction with other drugs, can be used to treat diseases or conditions discussed herein. For example, to treat a particular disease or condition, the siRNA molecules can be administered to a subject or can be administered to other appropriate cells evident to those skilled in the art, individually or in combination with one or more drugs under conditions suitable for the treatment.

In a further embodiment, the siRNA molecules can be used in combination with other known treatments to treat conditions or diseases discussed above. For example, the described molecules could be used in combination with one or more known therapeutic agents to treat a disease or condition. Non-limiting examples of other therapeutic agents that can be readily combined with a siRNA molecule of the invention are enzymatic nucleic acid molecules, allosteric nucleic acid molecules, antisense, decoy, or aptamer nucleic acid molecules, antibodies such as monoclonal antibodies, small molecules, and other organic and/or inorganic compounds including metals, salts and ions.

In one embodiment, the invention features an expression vector comprising a nucleic acid sequence encoding at least one siRNA molecule of the invention, in a manner which allows expression of the siRNA molecule. For example, the vector can contain sequence(s) encoding both strands of a siRNA molecule comprising a duplex. The vector can also contain sequence(s) encoding a single nucleic acid molecule that is self complementary and thus forms a siRNA molecule. Non-limiting examples of such expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi: 10.1038/nm725.

In another embodiment, the invention features a mammalian cell, for example, a human cell, including an expression vector of the invention.

In yet another embodiment, the expression vector of the invention comprises a sequence for a siRNA molecule having complementarity to a RNA molecule referred to by a Genbank Accession numbers, for example BACE genes such as Genbank Accession Nos. NM_{—}012104 (BACE), NMl 006222 (PIN-1), L76517 (PS-1) and/or L43964 (PS-2).

In one embodiment, an expression vector of the invention comprises a nucleic acid sequence encoding two or more siRNA molecules, which can be the same or different.

In another aspect of the invention, siRNA molecules that interact with target RNA molecules and down-regulate gene encoding target RNA molecules (for example target RNA molecules referred to by Genbank Accession numbers herein) are expressed from transcription units inserted into DNA or RNA vectors. The recombinant vectors can be DNA plasmids or viral vectors. siRNA expressing viral vectors can be constructed based on, but not limited to, adeno-associated virus, retrovirus, adenovirus, or alphavirus. The recombinant vectors capable of expressing the siRNA molecules can be delivered as described herein, and persist in target cells. Alternatively, viral vectors can be used that provide for transient expression of siRNA molecules. Such vectors can be repeatedly administered as necessary. Once expressed, the siRNA molecules bind and down-regulate gene function or expression via RNA interference (RNAi). Delivery of siRNA expressing vectors can be systemic, such as by intravenous or intramuscular administration, by administration to target cells ex-planted from a subject followed by reintroduction into the subject, or by any other means that would allow for introduction into the desired target cell.

Pharmaceutical composition comprising formulations containing a dsRNA of the invention are also encompassed by the invention. The pharmaceutical composition may be administered in a dosage sufficient to inhibit expression of the target gene.

As used herein, a “pharmaceutical composition” comprises a pharmacologically effective amount of a dsRNA and a pharmaceutically acceptable carrier. As used herein, “pharmacologically effective amount,” “therapeutically effective amount” or simply “effective amount” refers to that amount of an siRNA effective to produce the intended pharmacological, therapeutic or preventive result. For example, if a given clinical treatment is considered effective when there is at least a 50% reduction in a measurable parameter associated with a disease or disorder, a therapeutically effective amount of a drug for the treatment of that disease of disorder is the amount necessary to effect that at least 50% reduction.

The term “pharmaceutically acceptable carrier” refers to a carrier for administration of a therapeutic agent. Such carriers are well known to one skilled in the art.

Therapeutic kits are also envisioned by the inventors. The kits comprise the reagents, agents, and materials that may be required to practice the above methods of the invention, including, but not limited to those reagents necessary for transfection or transformation of cells with siRNA. Such kits may also comprise siRNA made by the methods of the present invention. The kits will generally contain, in suitable container means, a pharmaceutically acceptable formulation of siRNA. The kit may have a single container means, and/or it may have distinct container means for each compound or each reaction mixture or step.

### (II) Antisense Embodiments

The present invention also encompasses various methods and compositions for inhibiting the transcription of the HDAC 11 gene. Similarly, the invention also provides methods and compositions for inhibiting the translation of HDAC 11 mRNA into protein.

For example, in one approach, a method of inhibiting the transcription of the HDAC 11 gene comprises contacting the HDAC 11 gene with a HDAC 11 antisense polynucleotide. In yet another embodiment, there is provided a method of inhibiting HDAC 11 in RNA translation comprising contacting the HDAC 11 mRNA with an antisense polynucleotide. An alternative approach proposes a HDAC 11 specific ribozyme to cleave the HDAC 11 message, thereby inhibiting translation. Such antisense and ribozyme based methods can also be directed to the regulatory regions of the HDAC 11 gene, such as the HDAC 11 promoter and/or enhancer elements. The use of antisense and ribozyme molecules to inhibit transcription and translation is well known in the art.

Other factors that inhibit the transcription of HDAC 11 are also useful to treat cancers expressing HDAC 11. Similarly, factors/agent that specifically interfere with HDAC 11 mediated processes, e.g. deacetylation etc. are also useful to treat cancers. Thus, cancer treatment methods utilizing such factors/agents also fall within the scope of the invention.

Representative, nucleic acid related embodiments of the invention disclosed herein are genomic DNA, cDNAs, ribozymes, and antisense molecules, as well as nucleic acid molecules based on an alternative backbone, or including alternative bases, whether derived from natural sources or synthesized, and include molecules capable of inhibiting the RNA or protein expression of HDAC 11.

Antisense RNA technology has been developed as an approach to inhibiting gene expression. An “antisense” RNA molecule is one which contains the complement of, and can therefore hybridize with, messenger RNAs of the cell. It is widely believed that the hybridization of antisense RNA to its cellular RNA complement effectively prevents expression of the cellular RNA, perhaps by limiting its translatability. While various studies have involved the processing of RNA or direct introduction of antisense RNA oligonucleotides to cells for the inhibition of gene expression (Brown, et al., 1989; Wickstrom, et al., 1988; Smith, et al., 1986; Buvoli, et al., 1987), the more common means of cellular introduction of antisense RNAs has been through the construction of recombinant vectors which will express antisense RNA once the vector is introduced into the cell.

A principle application of antisense RNA technology has been in connection with attempts to affect the expression of specific genes. Antisense technology has also been applied in attempts to inhibit the expression of various oncogenes. For example, Kasid, et al., 1989, report the preparation of recombinant vector construct employing Craf-1 cDNA fragments in an antisense orientation, brought under the control of an adenovirus 2 late promoter. According to the report, the introduction of the recombinant construct into a human squamous carcinoma resulted in a greatly reduced tumorigenic potential relative to cells transfected with control sense transfectants. Similar results have been reported in the use of Cmyc antisense constructs which were effective to differentiate and inhibit G-1 progression in Friend Murine Erythroleukemia cells. See Prochownik, et al., 1988. Therefore, it appears that antisense technology shows potential promise as a means of external control of gene expression.

A representative method of the invention encompasses antisense molecules that may be RNAs or other molecules, including peptide nucleic acids (PNAs) or non-nucleic acid molecules such as phosphorothioate derivatives, that specifically bind DNA or RNA in a base pair-dependent manner. Applicants note that a skilled artisan may readily obtain these classes of nucleic acid molecules using the nucleic acid sequence of human HDAC 11) (SEQ ID NO: 1) polynucleotides sequences disclosed herein.

In general, antisense technology entails the administration of exogenous oligonucleotides that bind to a target polynucleotide located within the cells. The term “antisense” refers to the fact that such oligonucleotides are complementary to their intracellular targets sequences, specifically human HDAC 11 of SEQ ID NO: 1 including variants and fragments thereof. Refer to Jack Cohen, Oligodeoxynucleotides, Antisense Inhibitors of Gene Expression, CRC Press, 1989; and Synthesis 1:1-5 (1988). The HDAC 11 specific antisense oligonucleotides of the present invention include derivatives detailed in Jack Cohen, supra, such as phosphorothioate derivatives or S-oligonucleotides which are believed to exhibit enhanced cancer cell growth inhibitory action. As noted is said reference, S-oligos (nucleoside phosphorothioates) are isoelectronic analogs of an oligonucleotide (O-oligo) in which a nonbridging oxygen atom of the phosphate group is replaced by a sulfur atom. The S-oligos of the present invention can be prepared by treatment of the corresponding O-oligos with 3H-1,2-benzodithiol-3-one-1,1-dioxide, which is a sulfur transfer reagent. See Iyer, R. P. et al., J. Org. Chem. 55:4693-4698 (1990); and Iyer, R. P. et al., J. Am. Chem. Soc. 112:1253-1254 (1990).

Other HDAC 11 specific antisense oligonucleotides of the present invention may include morpholino antisense oligonucleotides known in the art (see, e.g., Partridge et al., 1996, Antisense & Nucleic Acid Drug Development 6: 169-175).

The HDAC 11 specific antisense oligonucleotides of the present invention typically can be RNA or DNA that is complementary to and stably hybridizes with the first 100 5′ codons or last 100 3′ codons of the HDAC 11 specific genomic sequence or the corresponding mRNA. Absolute complementarity is not required, although high degrees of complementarity are preferred. Use of an oligonucleotide complementary to this region allows for the selective hybridization to HDAC 11 specific n3RNA and not to mRNA specifying other proteins. In one embodiment, HDAC 11 specific antisense oligonucleotides of the present invention are 15 to 30-mer fragments of the antisense DNA molecule that have a sequence that hybridizes to HDAC 11 specific mRNA. Optionally, HDAC 11 specific antisense oligonucleotide is a 30-mer oligonucleotide that is complementary to a region in the first 10 5′ codons or last 10 3′ codons of HDAC 11 specific. Alternatively, the antisense molecules are modified to employ ribozymes in the inhibition of HDAC 11 specific expression, see, e.g., L. A. Couture & D. T. Stinchcomb; Trends Genet. 12: 510-515 (1996).

### (III) Inhibition of HDAC 11 Specific Protein Function

The invention also provides various methods and compositions for inhibiting the biological, enzymatic functions of specific HDAC molecules or its association with other protein(s) as well as methods for inhibiting HDAC 11 specific function.

(A) Inhibition of HDAC 11 Specific with Intracellular Antibodies

Cancer is known to be a multistep process where cellular growth becomes progressively dysregulated and cells progress from a normal physiological state to precancerous and then cancerous states (see, e.g., Alers et al., Lab Invest. 77(5): 437-438 (1997) and Isaacs et al., Cancer Surv. 23: 19-32 (1995)). In this context, upon presentation of clinical signs of colon, cervical or lung cancer, examination of a biological sample for evidence of dysregulated cell growth, will allow for early detection of such aberrant physiology, before a pathologic state such as cancer has progressed to a stage that therapeutic options are more limited and or the prognosis is worse.

In such examinations, the status of various markers for one of lung, colon or cervical cancer in a biological sample of interest can be compared, for example, to the status of the same markers in a corresponding normal sample (e.g. a sample from that individual or alternatively another individual that is not affected by a pathology). An alteration in the status of said markers in the biological sample (as compared to the normal sample) provides evidence of dysregulated cellular growth. In addition to using a biological sample that is not affected by a pathology as a normal sample, one can also use a predetermined normative value such as a predetermined normal level of mRNA expression to compare the marker status in a sample.

The term “status” in this context is used according to its art accepted meaning and refers to the condition or state of a gene and its products. Typically, skilled artisans use a number of parameters to evaluate the condition or state of a gene and its products. These include, but are not limited to the location of expressed gene products (including the location of the marker expressing cells etc) as well as the level, and biological activity of expressed gene products including HDAC 11.

Consequently, HDAC 11 proteins of SEQ ID NO: 2 or fragments or immunologically active fragments there have a number of different uses including generating antibodies. Antibodies raised against an HDAC protein or fragment thereof are useful in the management of human cancers characterized by aberrant cell growth. Thus, for patients having or presenting symptoms consistent with any one of more of the above mentioned cancers, early intervention by way of inhibiting HDAC 11 protein function or expression level may present an amicable means of early intervention notwithstanding the observations of the inventors that in said cell proliferative disorders the expression levels of HDAC 11 is normal or near normal. Thus, an aspect of the invention provides antibodies that bind to HDAC 11 proteins or related proteins in a manner specific to abrogate the biological proteins attendant said proteins, e.g., enzymatic activities such as deacetylation etc. Preferred antibodies specifically bind to HDAC 11 and do not bind (or bind weakly) to peptides or proteins that are not HDAC 11.

In another approach, a recombinant vector that encodes single chain antibodies that specifically bind to HDAC 11 are introduced into HDAC 11 expressing cells previously correlated to a specific cancer, via gene transfer technologies. Accordingly, the encoded single chain ant-HDAC 11 antibody is expressed intracellularly, binds to HDAC 11 proteins and thereby inhibits its function. Methods for engineering such intracellular single chain antibodies are well known. Such intracellular antibodies, also known as “intrabodies”, are specifically targeted to a particular compartment within the cell, providing control over where the inhibitory activity of the treatment is focused. This technology has been successfully applied in the art (for review, see Richardson and Marasco, 1995, TIBTECH vol. 13).

Various methods for the preparation of antibodies are well known in the art. For example, antibodies can be prepared by immunizing a suitable mammalian host using a HDAC 111 protein, peptide, or fragment, in isolated or immunoconjugated form a (Antibodies: A Laboratory Manual, CSH Press, Eds., Harlow, and Lane (11988); Harlow, Antibodies, Cold Spring Harbor Press, NY (11989)).

(B) Inhibition of HDAC 11 with Recombinant Proteins

Recombinant molecules that specifically bind to HDAC 11 and thereby inhibit HDAC 11 specific function Eire also within the scope of the invention. For example, these recombinant-molecules specifically prevent or inhibit HDAC 11 mediated activity within a cell.

Screening Methods

Since HDAC 11 and its polymorphism variant play a role in transcription, chromosome stability, cell cycle progression, aging, regulation of neuronal phenotype, DNA replication and the response to DNA damage, and in view of the nexus found by the present inventors vis-à-vis normal expression levels of the protein of SEQ ID NO: 2 and certain cell proliferation disorders, the HDAC 11 protein, and respective nucleic acids can be used in screening essays to identify candidate agents or drugs that modulate HDAC bioactivity, for potential use to treat neoplastic disorders, for example, to kill cancer cells and tumor cells exhibiting uncontrolled cell growth for numerous reasons, e.g., the lack of a suppressor molecule such as p53. As well, HDAC 11 proteins and encoding nucleic acids, as well as the agents that modulate HDAC 11 activity or function, can be used as effectors in methods to regulate cell growth, e.g., to kill neoplastic cells.

Antagonists or inhibitors of HDAC 11 may be antibodies specific for the HDAC 11 protein that can be used directly as an antagonist, or indirectly as a targeting or delivery mechanism for bringing a pharmaceutical agent to cells or tissue which express the HDAC 11 protein. Other methods to inhibit the expression of the HDAC 11 protein include antisense and triple helix strategies as described herein.

Other antagonists or inhibitors of the HDAC 11 protein may be produced using methods which are generally known in the art, including the screening of libraries of pharmaceutical agents to identify those which specifically bind the HDAC 11 protein. The HDAC 11 protein, or fragment thereof, preferably its functional or immunogenic fragments, or oligopeptides related thereto, can be used for screening libraries of compounds in any of a variety of drug screening techniques. The fragment employed in such screening may be free in solution, affixed to a solid support, borne on a cell surface, or located intracellularly. The formation of binding complexes, between the HDAC 11 protein, or fragment thereof, or derivative thereof, and the agent being tested, may be measured using methods known in the art.

An alternative technique for drug screening provides for high throughput screening of compounds having suitable binding affinity to the HDAC 11 protein. See published PCT application WO84/03564.

Since the protein of SEQ ID NO:2 may deacetylate substrates, preferably acetylated histones, either directly or indirectly as enzymes cofactors, this property may be exploited to identify HDAC 11 inhibitors. The deacetylation activity of the protein of SEQ ID NO:2 or fragment thereof or may be assayed using any of a number of methods known to those skilled in the art.

The following techniques may be used to detect or assay acetylation or deacetylation. Such methods, when applicable, may be used for in vitro assays. A representative assay proposes treating a specific cell line with different concentrations of a candidate substance. Methods for measuring hyperacetylation of histones have been described in detail (Verdel and Khochbin, 1999; Fischle et al., 1999; Grozinger et al., 1999) and are known in the art. For example, the appearance of hyperacetylated H3histone can be monitored using antibody raised against hyperacetylated histone H3 and detected by cytofluorimetric measurement of immunofluorescence (see, Van Lint et al., 1996). Alternatively, cells may be lysed, the histones purified and analyzed on a Triton/acid/urea gel. Analytical ultracentrifugation is often used also to detect histone acetylation.

Acetylated substrates used in such methods are preferably acetylated histones and acetyltransferases. Measuring the rate of deacetylation of [³H]-labeled-acetylated histones also is a useful assay in the present invention. For example, the HDAC 11 protein or a fragment thereof is added to a sample containing a substrate under conditions favoring deacetylation, and allowed to catalyze the deacetylation of the substrate. In a preferred embodiment, the deacetylation is carried out using a standard assay such as those described in Landry and collaborators. Refer to Landry et al., Proc. Natl. Acad. Sci. 97:5807-5811 (2000), the disclosure of which is incorporated by reference in its entirety. Deacetylated histones obtained by this method may be mixed with purified naked DNA (plasmid preparations for example) in order to reconstitute chromatine-like structures in vitro. Such structures are of great interest in the study of enzymatic factors involved in transcription and replication. Natural transcription factors are unable to enter the condensed chromatin, and the gene function is effectively switched-off.

A representative method according to the invention is directed to a method for determining the ability of a candidate substance to inhibit HDAC 11 activity; generally including the steps of:

(a) providing a source of HDAC 11 enzyme;

(b) contacting the enzyme with a candidate substance;

(c) determining the enzyme function in step (b); and

(d) comparing the enzyme function in step (c) with the enzyme function of the enzyme in the absence of the candidate substance, wherein increased enzyme function in the presence of the candidate substance, as compared to enzyme function in the absence of the candidate substance, identifies the candidate substance as an inhibitor of cell proliferation.

An exemplary embodiment proposes a method for screening test agents to identify modulating agents which inhibit or antagonize deacetylation activity of a histone deacetylase, proposes:
(i) combining an isolated polypeptide of SEQ ID NO:2 or polymorphic or biologically active variants thereof having a histone deacetylase activity with a histone deacetylase substrate and a test agent in a reaction mixture; and
(ii) determining the conversion of the substrate to product; wherein a statistically significant decrease in the conversion of the substrate in the presence of the test agent indicates identification of a modulating agent which inhibits or antagonizes the deacetylation activity of histone deacetylase.

Also contemplated are methods for screening test agents to identify modulating agents that inhibit or antagonize interaction of histone deacetylase with a histone deacetylase binding protein. This method proposes: (i) combining a HDAC 11 polypeptide having a histone deacetylase activity with the histone deacetylase binding protein and a test agent in a reaction mixture; and (ii) detecting the interaction of the polypeptide with the histone deacetylase binding protein to form a complex; wherein a statistically significant decrease in the interaction of the polypeptide and protein in the presence of the test agent indicates identification of a modulating agent which inhibits or antagonizes interaction of the HDAC 11 polypeptide with the histone deacetylase binding protein.

High Throughput Screening—Test compounds can be screened for the ability to bind to HDAC 11 polypeptides or polynucleotides or to affect HDAC 11 activity or HDAC 11 gene expression using high throughput screening. Using high throughput screening, many discrete compounds can be tested in parallel so that large numbers of test compounds can be quickly screened. The most widely established techniques utilize microtiter plates. The wells of the microtiter plates typically require assay volumes that range from 2 to 500 μl. In addition to the plates, many instruments, materials, pipettors, robotics, plate washers, and plate readers are commercially available to fit the microtiter format.

Yet another example is described by Salmon et al., Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813, which proposes placing test samples in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Other assays include binding assays, functional assays etc which are well known to one skilled in the art.

It is recognized that the HDAC 11 therapeutics, e.g., HDAC 11 inhibitor molecules (HDAC 11i) contemplated by the present invention, e.g., antisense molecules, anti-HDAC antibodies, or antibody fragments, small molecules, (chemical moieties, or dsRNA constructs (siRNA molecules) of the invention can be used in combination with other HDAC inhibitory agents, e.g., trichostatin A (D. M. Vigushin et al., 2001, Clin. Cancer Res. 7(4):971-6); trapoxin A (Itazaki et al., 1990, J. Antibiot. 43:1524-1532), MS-275 (T. Suzuki et al., 1999, J. Med. Chem. 42(15):3001-3), CHAP (Y. Komatsu et al., 2001, Cancer Res. 61(11):4459-66), CI-994 (see, e.g., P. M. LoRusso et al., 1996, New Drugs 14(4):349-56), SAHA (V. M. Richon et al., 2001, Blood Cells Mol. Dis. 27(1):2604), depsipeptide (FR901228; FK228; V. Sandor et al., 2002, Clin. Cancer Res. 8(3):718-28), CBHA (D. C. Coffey et al., 2001, Cancer Res. 61 (9):3591-4), pyroxamide, (L. M. Butler et al, 2001, Clin. Cancer Res. 7(4):962-70), CHAP31 (Y. Komatsu et al., 2001, Cancer Res. 61(11):4459-66), HC-toxin (Liesch et al., 1982, Tetrahedron 38:4548), chlamydocin (Closse et al., 1974, Helv. Chim. Acta 57:533-545), Cly-2 (Hirota et al., 1973, Agri. Biol. Chem. 37:955-56), WF-3161 (Umehana et al., 1983, J. Antibiot. 36, 478-483; M. Kawai et al., 1986, J. Med. Chem. 29(11):2409-11), Tan-1746 (Japanese Pat. No. 7196686 to Takeda Yakuhin Kogyo KK), apicidin (S. H. Kwon et al., 2002, J. Biol. Chem. 277(3):2073-80), as LAQ824 (Remiszewski, SW Curr. Med. Chem. 10, 2393, 2003), PXD101 (Plumb et al., Mol. Cancer. Ther. 2, 721, 2003), FK228 (Yoshida et al., Curr. Med. Chem. 10, 2351, 2003), MS27-275 (Saito et al., Proc. Natl. Acad. Sci. USA 96, 4592, 1999) and CI994 (Kraker et al., Mol. Cancer. Ther. 2, 401, 2003) and analogs thereof.

Kits

For use in the therapeutic applications contemplated herein, kits are also within the scope of the invention. Such kits can comprise a carrier, package or container that is compartmentalized to receive one or more containers such as vials, tubes, and the like, each of the container(s) comprising one of the separate elements to be used in the method. For example, the container(s) can comprise an antisense construct with detectable label. Alternatively, the container may comprise a probe that is or can be detectably labeled. Such probes can be an antibody or polynucleotide specific for an HDAC 11 specific-related protein or a HDAC 11 specific gene or message, respectively. The kit can include all or part of the amino acid sequence of SEQ ID NO: 2 or analogs thereof, or a nucleic acid molecules that encodes such amino acid sequences.

A label can be present on the container to indicate that the composition is used for a specific therapy or non-therapeutic application, and can also indicate directions for either in vivo or in vitro use, such as those described above. Directions and or other information can also be included on a label or on an insert which is included with the kit.

General Considerations for Therapeutic Strategies

Gene transfer and gene therapy technologies can be used to deliver therapeutic polynucleotide molecules i.e., antisense or dsRNA i molecules to 0 cancer cells or tissue. Synthesizing HDAC 11 specific (i.e., antisense, ribozyme, RNAi or polynucleotides encoding intrabodies and other HDAC 11 specific inhibitory molecules). A number of gene therapy approaches are known in the art. For example, recombinant vectors encoding HDAC 11 specific antisense polynucleotides, ribozymes, factors capable of interfering with HDAC 11 specific transcription, and so forth, can be delivered to target tumor cells using such gene therapy approaches. Other techniques including those detailed in the Examples below may also be used to deliver the HDAC therapeutics to the target cell or tissue.

Importantly, the aforementioned therapeutic approaches can be combined with any one of a wide variety of surgical, chemotherapy or radiation therapy regimens. The therapeutic approaches of the invention can enable the use of reduced dosages of chemotherapy (or other therapies) and/or less frequent administration, an advantage for all patients and particularly for those that do not tolerate the toxicity of the chemotherapeutic agent well.

The anti-tumor activity of a particular composition (e.g., antisense, ribozyme, intrabody, small molecule inhibitor), or a combination of such compositions, can be evaluated using various in L vitro and in vivo assay systems. In vitro assays that evaluate therapeutic activity include cell growth assays, soft agar assays and other assays indicative of tumor promoting activity, binding assays capable of determining the extent to which a therapeutic composition will inhibit the binding of HDAC 11 specific to a binding partner, etc.

In vivo, the effect of a HDAC 11 specific therapeutic composition may be evaluated in a suitable animal model. For example, xenogenic lung cancer or colon cancer models can be used, wherein human lung or colon cancer explants or passaged xenograft tissues are introduced into immune compromised animals, such as nude or SCID mice (Shibayama et al., 1991, J Urol., 146(4):1136-7; Beecken et al., 2000, Urology, 56(3):521-526). Indeed, the overall efficacy can be predicted using assays that measure inhibition of tumor formation, tumor regression or metastasis, and the like.

In vivo assays that evaluate progression of apoptosis are useful in evaluating therapeutic compositions. In an exemplary embodiment, xenografts from tumor bearing mice treated with an HDAC 11 therapeutic can be examined for the presence of apoptotic foci and compared to untreated control xenograft-bearing mice. The extent to which apoptotic foci are found in the tumors of the treated mice, in turn, may provide an indication of the therapeutic efficacy of the composition.

The HDAC 11 therapeutic compositions identified herein can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. See, Remington's Pharmaceutical Sciences 16.sup.th Edition, A. Osal., Ed., 1980).

Dosages and administration protocols for the treatment of cancers using the foregoing methods will vary with the method and the target cancer and will generally depend on a number of other factors appreciated in the art.

### Example 1

### Materials and Methods

siRNA Design.

Sequences of the type AA(N₁₉)dTdT (N, any nucleotide) from the targeted mRNA were designed based on the rules suggested by Elbashir et al. (Genes Dev, 2001) and purchased from Dharmacon Research or Oligo Engine as annealed, deprotected, double-stranded 21mers. The N₁₉ sequences targeting HDAC11 mRNA corresponded to the following nucleotide positions relative to the Genbank accession number NM_{—}024827: HDAC11.2 nt. 513-531; HDAC11.3 nt. 582600; HDAC11.4 nt. 1032-1050; HDAC11.5 nt. 1344-1362.

Representative dsRNA constructs for specifically silencing human HDAC 11 via RNA interference include:

| | | |
|---|---|---|
| 11.2 | | |
| 1 | AAGUUUCUGU UUGAGCGUGU G | (SEQ ID NO: 3) |
| | CAAAGACA AACUCGCACA CAA | (SEQ ID NO: 7) |
| 11.3 | | |
| 1 | AAUGGGCAUG AGCGAGACUU AAC | (SEQ ID NO: 4) |
| | ACCCGUAC UCGCUCUGAA UUGAA | (SEQ ID NO: 8) |
| 11.4 | | |
| 1 | AACUCAGACA CACCGCUGCU U | (SEQ ID NO: 5) |
| | GAGUCUGU GUGGCGACGA AAA | (SEQ ID NO: 9) |
| 11.5 | | |
| 1 | AACUGAGAAU UGGAGAGGAC A | (SEQ ID NO: 6) |
| | GACUCUUA ACCUCUCCUG UAA | (SEQ ID NO: 10) |

Construct 11.2 targets nucleotides 511-531 of human HDAC gene, i.e. SEQ ID NO: 1, comprising the nucleotide sequence aagmctgtttgagcgtgtg.

Construct 11.3 targets nucleotides 580-600 of human HDAC gene, i.e. SEQ ID NO: 1, comprising the nucleotide sequence aatgggcatgagcgagacttc.

Construct 11.4 targets nucleotides 1030-1050 of human HDAC gene, i.e. SEQ II) NO: 1, comprising the nucleotide sequence aactcagacacaccgctgctt.

Construct 11.5 targets nucleotides 1342-1362 of human HDAC gene, i.e. SEQ ID NO: 1, comprising the nucleotide sequence aactgagaattggagaggaca.

Cell culture and siRNA Transfection.

HeLa human cervical carcinoma cell line was grown in Dulbecco modified Eagle medium (DMEM, Gibco); HCT-116 colorectal carcinoma cells were grown in McCoy's SA medium (Gibco); A549 human lung carcinoma cells were cultured in F12K Nutrient Mixture (Gibco). All media were supplemented with 10% fetal bovine serum (PBS, Gibco), 2 mM L-glutamine (Gibco), 100 U of penicillin per ml and 100 μg of streptomycin per ml (Gibco).

The day before transfection cells were trypsinized and transferred to 6-well plates at 70-80% confluency, in a final volume of 2 ml. Transfection was performed by using lipofectamine 2000 (Invitrogen) according to manufacturer's instructions with minor modifications. Briefly, a few hours before transfection, culture medium was replaced with 1 ml of fresh medium supplemented with 12% FBS without antibiotics. Lipofectamine 2000 (4 μl per well) was incubated with 100 μl of Optimem 1 medium (Gibco) for 5 minutes at room temperature; in the meantime 3 μl of 20 μM siRNA were diluted in another 100 μl of Optimem 1. The two mixtures were then mixed and incubated for 25 minutes at room temperature. The resulting final mixture was added to the cells of a 6-well plate to have a final siRNA concentration of 50 nM. Transfection was carried out for 4 hours, then transfection mixture was removed, cells were washed, trypsinized, and replated at different concentrations for the different assays. Silencing was assayed 48 hours post transfection by western blot and Taqman analysis.

### Immunoblotting.

A rabbit polyclonal antibody was raised against a peptide corresponding to the first 15 amino acids of the open reading frame of HDAC11 (MLHTTQLYQHVPETR) (SEQ ID NO: 14). For Western blot analysis, transfected cells were washed once with phosphate-buffered saline (PBS) and lysed directly in the culture dish with 150 μl per well of 1% SDS in PBS. Protein extracts were sheared with Qiashredder columns (Qiagen). Samples were diluted in sample buffer, heated for 5 min at 95° C., electrophoresed on 10% SDS-polyacrylamide gels and blotted onto a nitrocellulose membrane. Primary antibody against HDAC11 was diluted 1:5000 in blocking buffer and incubated at 4° C. overnight. A peroxidase-conjugated secondary antibody (Pierce) diluted 1:5000 in blocking buffer was incubated 1 hour at room temperature and detection was carried out using the SuperSignal West Pico Chemiluminescent Substrate (Pierce). To confirm equal loading, the filter was then reprobed with an anti-GAPDH as the primary antibody, diluted 1:5000 in blocking buffer and incubated at 4° C. overnight. An alkaline phosphatase-conjugated secondary antibody was used at a 1:2000 dilution in blocking buffer, and revealed with NBT and BCIP substrates.

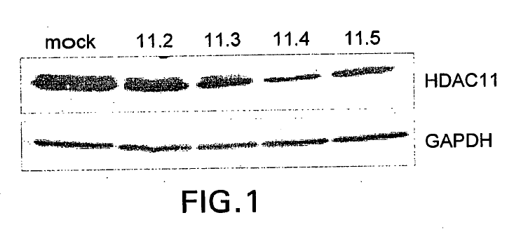

The results are disclosed in FIG. 1 which details the gene knock down of HDAC11 in human cells. The upper panel details the results of a western blot analysis of HCT116 cells transfected with 4 different siRNA constructs, e.g., 11.2, 11.3, 11.4, 11.5 each of which is directed against a specified region within the HDAC11 mRNA 48 hours post-transfection.

### Cell Growth Assay.

After 4 hours of transfection, cells were washed, trypsinized and replated in 96-well Cytostar-T scintillating microplates at 2500 or 5000 cells per well in triplicates. 80 nCi of methyl-¹⁴C-thymidine (Amersham Pharmacia) were added to each well. Labeled thymidine incorporation was measured with a TOP Count NXT Microplate Scintillation and Luminescence Counter (Packard) every 8-12 hours up to 72 hours post transfection.

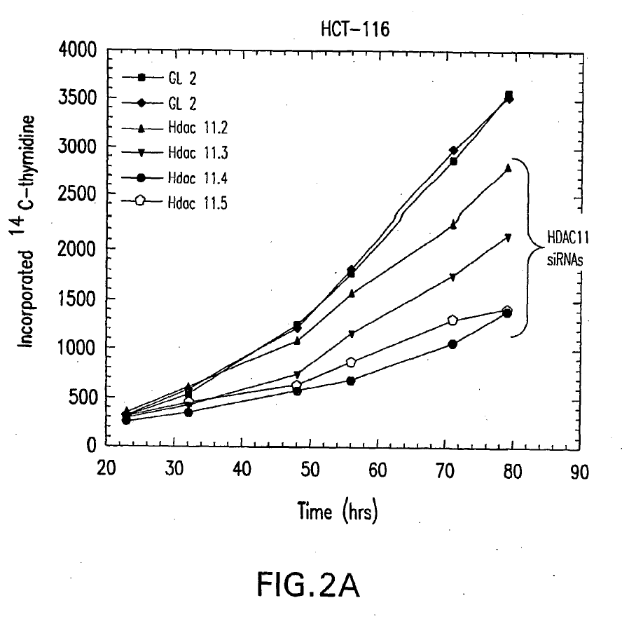

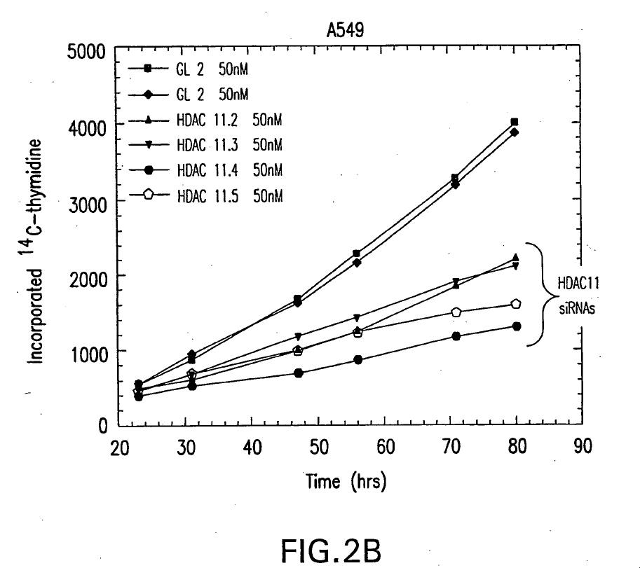

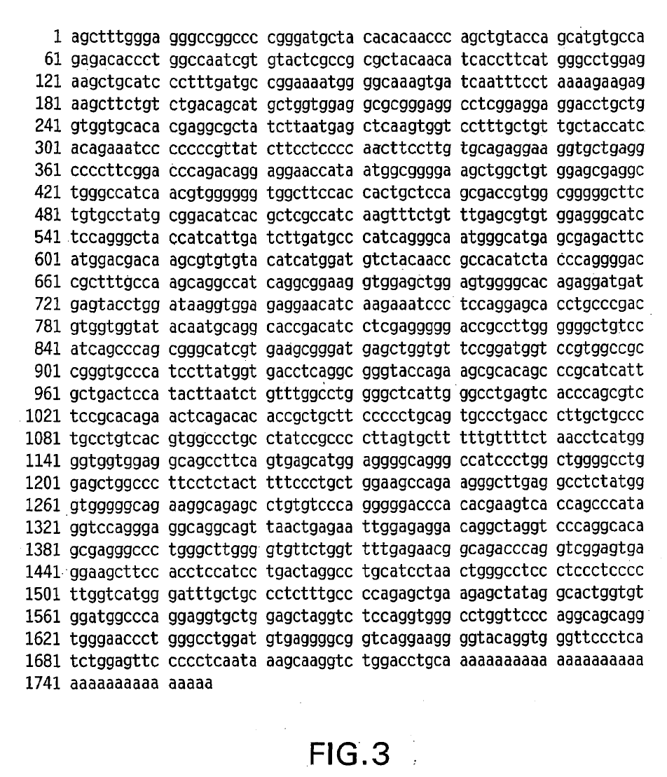

FIGS. 2a and 2b, detail the cell growth curves of HCT116 or A549 cells transfected with siRNA constructs of the invention directed against HDAC11 or GL2 (Control).

### RNA Extraction and Taqman Analysis.

At the indicated time points transfected cells were quickly washed once in PBS and total RNA was extracted using the Rneasy kit (Qiagen), according to manufacturer's instructions. A Dnase I treatment was added to the basic extraction protocol, as suggested by the manufacturer. RNAs were quantified by spectrophotometric absorbance and their quality was analyzed on 1% denaturing gel.

Quantitative RT-PCR of HDAC11 mRNA was performed in triplicate on 100 ng of total RNA per well, by using the One Step RT-PCR Master Mix (Applied Biosystem) with the following set of primers and probe: primer sense: 5′-CCTCAGGCGGGTACCAGAA-3′ (SEQ ID NO: 11), 200 nM; primer antisense: 5′-CAGGCCAAACAGATTAAGTATGGA-3′, (SEQ ID NO: 12) 400 nM; probe (FAM-TAMRA): 5′-CGCACAGCCCGCATCATTGCT-3′ (SEQ ID NO: 13), 150 nM. Normalization was done on the same amount of template by amplification of human GAPDH or human β-actin using the corresponding Pre-Developed Taqman Assay Reagents (Applied Biosystem), in triplicate, in the same plate as HDAC11. Detection was performed with an ABI Prism 7900HT Sequence Detection System.

HDAC11NM_024827
HDAC11.1
(SEQ ID NO: 15)
AA GUGGUCCUUUGCUGUUGCU
nt. 274-294 % GC = 47.6 Tm = 52.9
HDAC11.2
(SEQ ID NO: 16)
AA GUUUCUGUUUGAGCGUGUG
nt. 511-531 % GC = 42.9 Tm = 47
HDAC11.3
(SEQ ID NO: 17)
AA UGGGCAUGAGCGAGACUUC
nt. 580-600 % GC = 52.4 Tm = 53.6 DG dimer = -2.1
HDAC11.4
(SEQ ID NO: 18)
AA CUCAGACACACCGCUGCUU
nt. 1030-1050 % GC = 52.4 Tm = 54.7 DG loop = -1.
HDAC11.5
(SEQ ID NO: 19)
AA CUGAGAAUUGGAGAGGACA
nt. 1342-1362 % GC = 42.9 Tm = 49.2

1. (canceled)

2. (canceled)

3. A short interfering nucleic acid (siRNA) molecule that down regulates expression of a mammalian HDAC 11 gene by RNA interference, wherein said siRNA molecule comprises a first sequence and a second sequence, wherein the first sequence comprises a sequence of nucleotides as set forth in one of SEQ ID NOs: 3-6 and the second sequence comprises a sequence of nucleotides as set forth in SEQ ID NOs: 7-10.

4. A method for reducing the expression of a target gene in a population of cells, the method comprising: introducing the short interfering nucleic acid of claim 3 into said population of cells in an amount effective to down regulate expression of the target gene via RNA interference.

5. The siRNA molecule of claim 3, wherein said siRNA molecule is adapted for use to treat a cell proliferative disorder, wherein said disorder is one of colon cancer, cervical cancer or lung cancer.

6. The siRNA molecule of claim 3, wherein said siRNA molecule comprises a sense region and an antisense region and wherein said antisense region comprises sequence complementary to an RNA sequence encoding human HDAC 11 and the sense region comprises sequence complementary to the antisense region.

7. The siRNA molecule of claim 3, wherein said siRNA molecule is assembled from two nucleic acid fragments wherein one fragment comprises the sense region and the second fragment comprises the antisense region of said siRNA molecule.

8. The siRNA molecule of claim 3, wherein said sense region and antisense region are covalently connected via a linker molecule.

9. A method for attenuating the expression of a target gene in a cell comprising: introducing the siRNA of claim 3 into the cell in an amount sufficient to attenuate expression of the target gene, wherein the sense and antisense regions comprise about 19-23 nucleotides, and wherein said antisense region of said siRNA comprises a sequences of nucleotides that are substantially homologous to a region of the RNA sequence encoding human HDAC 11.

10. The method according to claim 9, wherein said region in siRNA comprises a sequences of nucleotides that are homologous to a sequence of nucleotides as set forth in SEQ ID NO: 1.

11. The method according to claim 10, wherein said siRNA molecule comprises a sequences of nucleotides that are homologous to a sequence of nucleotides selected from the group consisting of nucleotides 513-531; 582-600; 1032-1050; and 1344-1362 of SEQ ID NO:1.

12. A method for the treatment of a cell proliferative disorder, the method comprising administering to a subject in need thereof an agent which inhibits the bioactivity of human HDAC 11 protein or an agent which decreases expression of an HDAC 11 encoding gene, wherein said agent comprises the short interfering nucleic acid molecule of claim 3.

13. A method for inhibiting the growth or proliferation of a neoplastic cell, comprising contacting a tumor cell with an agent which inhibits the activity of HDAC 11 or decreases expression of a HDAC 11 encoding gene in an amount sufficient in induce cell-cycle arrest, wherein said agent comprises the short interfering nucleic acid of claim 3.

14. A method of screening for an agent which inhibits cell proliferative disorders, the method comprising testing a putative agent for the ability to inhibit HDAC 11 bioactivity or decrease expression of a HDAC 11 encoding gene.

15. The method according to claim 14, wherein the agent is selected from the group consisting of a RNAi construct targeted for silencing HDAC 11 gene expression, an HDAC 11 antisense oligonucleotide, a ribozyme targeted against HDAC 11, an antibody specific for HDAC 11, a ssDNA targeted against HDAC 11 dsDNA effective to form a triplex with the HDAC 11 dsDNA, or a chemical moiety effective to inhibit HDAC 11 function or activity.

16. The method according to claim 15, wherein said chemical moiety inhibits the function of said HDAC 11 protein by inhibiting the deacetylation activity attendant a native HDAC 11 protein.

17. (canceled)


SEQUENCE LISTING (19 sequences)

SEQ ID NO: 1 - DNA, 1755 bp, Homo sapiens
agctttgggagggccggccccgggatgctacacacaacccagctgtaccagcatgtgccagagacaccctggccaatcgtgtactcgccgcgctacaacatcaccttcatgggcctggagaagctgcatccctttgatgccggaaaatggggcaaagtgatcaatttcctaaaagaagagaagcttctgtctgacagcatgctggtggaggcgcgggaggcctcggaggaggacctgctggtggtgcacacgaggcgctatcttaatgagctcaagtggtcctttgctgttgctaccatcacagaaatcccccccgttatcttcctccccaacttccttgtgcagaggaaggtgctgaggccccttcggacccagacaggaggaaccataatggcggggaagctggctgtggagcgaggctgggccatcaacgtggggggtggcttccaccactgctccagcgaccgtggcgggggcttctgtgcctatgcggacatcacgctcgccatcaagtttctgtttgagcgtgtggagggcatctccagggctaccatcattgatcttgatgcccatcagggcaatgggcatgagcgagacttcatggacgacaagcgtgtgtacatcatggatgtctacaaccgccacatctacccaggggaccgctttgccaagcaggccatcaggcggaaggtggagctggagtggggcacagaggatgatgagtacctggataaggtggagaggaacatcaagaaatccctccaggagcacctgcccgacgtggtggtatacaatgcaggcaccgacatcctcgagggggaccgccttggggggctgtccatcagcccagcgggcatcgtgaagcgggatgagctggtgttccggatggtccgtggccgccgggtgcccatccttatggtgacctcaggcgggtaccagaagcgcacagcccgcatcattgctgactccatacttaatctgtttggcctggggctcattgggcctgagtcacccagcgtctccgcacagaactcagacacaccgctgcttccccctgcagtgccctgacccttgctgccctgcctgtcacgtggccctgcctatccgccccttagtgctttttgttttctaacctcatggggtggtggaggcagccttcagtgagcatggaggggcagggccatccctggctggggcctggagctggcccttcctctacttttccctgctggaagccagaagggcttgaggcctctatgggtgggggcagaaggcagagcctgtgtcccagggggacccacacgaagtcaccagcccataggtccagggaggcaggcagttaactgagaattggagaggacaggctaggtcccaggcacagcgagggccctgggcttggggtgttctggttttgagaacggcagacccaggtcggagtgaggaagcttccacctccatcctgactaggcctgcatcctaactgggcctccctccctccccttggtcatgggatttgctgccctctttgccccagagctgaagagctataggcactggtgtggatggcccaggaggtgctggagctaggtctccaggtgggcctggttcccaggcagcaggtgggaaccctgggcctggatgtgaggggcggtcaggaaggggtacaggtgggttccctcatctggagttccccctcaataaagcaaggtctggacctgcaaaaaaaaaaaaaaaaaaaaaaaaaaaaaaaaaaaa

SEQ ID NO: 2 - protein, 347 aa, Homo sapiens
MLHTTQLYQHVPETPWPIVYSPRYNITFMGLEKLHPFDAGKWGKVINFLKEEKLLSDSMLVEAREASEEDLLVVHTRRYLNELKWSFAVATITEIPPVIFLPNFLVQRKVLRPLRTQTGGTIMAGKLAVERGWAINVGGGFHHCSSDRGGGFCAYADITLAIKFLFERVEGISRATIIDLDAHQGNGHERDFMDDKRVYIMDVYNRHIYPGDRFAKQAIRRKVELEWGTEDDEYLDKVERNIKKSLQEHLPDVVVYNAGTDILEGDRLGGLSISPAGIVKRDELVFRMVRGRRVPILMVTSGGYQKRTARIIADSILNLFGLGLIGPESPSVSAQNSDTPLLPPAVP

SEQ ID NO: 3 - RNA, 21 nt, Artificial Sequence
  Completely synthetic oligonucleotide
aaguuucuguuugagcgugug

SEQ ID NO: 4 - RNA, 23 nt, Artificial Sequence
  Completely synthetic oligonucleotide
aaugggcaugagcgagacuuaac

SEQ ID NO: 5 - RNA, 21 nt, Artificial Sequence
  Completely synthetic oligonucleotide
aacucagacacaccgcugcuu

SEQ ID NO: 6 - RNA, 21 nt, Artificial Sequence
  Completely synthetic oligonucleotide
aacugagaauuggagaggaca

SEQ ID NO: 7 - RNA, 21 nt, Artificial Sequence
  Completely synthetic oligonucleotide
caaagacaaacucgcacacaa

SEQ ID NO: 8 - RNA, 23 nt, Artificial Sequence
  Completely synthetic oligonucleotide
acccguacucgcucugaauugaa

SEQ ID NO: 9 - RNA, 21 nt, Artificial Sequence
  Completely synthetic oligonucleotide
gagucuguguggcgacgaaaa

SEQ ID NO: 10 - RNA, 21 nt, Artificial Sequence
  Completely synthetic oligonucleotide
gacucuuaaccucuccuguaa

SEQ ID NO: 11 - DNA, 19 bp, Artificial Sequence
  Completely synthetic oligonucleotide
cctcaggcgggtaccagaa

SEQ ID NO: 12 - DNA, 24 bp, Artificial Sequence
  Completely synthetic oligonucleotide
caggccaaacagattaagtatgga

SEQ ID NO: 13 - DNA, 21 bp, Artificial Sequence
  Completely synthetic oligonucleotide
cgcacagcccgcatcattgct

SEQ ID NO: 14 - protein, 15 aa, Artifical Sequence
  Completely synthetic
MLHTTQLYQHVPETR

SEQ ID NO: 15 - protein, 12 aa, Homo sapiens
AAGGGCCGCGGC

SEQ ID NO: 16 - protein, 10 aa, Homo sapiens
GCGGAGCGGG

SEQ ID NO: 17 - protein, 15 aa, Homo sapiens
GGGCAGAGCGAGACC

SEQ ID NO: 18 - protein, 15 aa, Homo sapiens
CCAGACACACCGCGC

SEQ ID NO: 19 - protein, 16 aa, Homo sapiens
CGAGAAGGAGAGGACA